(19) United States
(12) Patent Application Publication
(10) Pub. No.: US 2009/0076475 A1
(43) Pub. Date: Mar. 19, 2009

(54) METHOD AND APPARATUS FOR DELIVERING THERAPEUTIC OXYGEN TREATMENTS

(75) Inventors: Julian T. Ross, McKinney, TX (US); Steven O. Dunford, Lewisville, TX (US)

Correspondence Address: OXYSURE SYSTEMS, INC, 10880 JOHN W. ELLIOT DR., SUITE 600, FRISCO, TX, 75034, US

(73) Assignee: OxySure Systems Inc., Frisco, TX (US)

(21) Appl. No.: 11/558,374

(22) Filed: Nov. 9, 2006

Related U.S. Application Data
(60) Provisional application No. 60/735,011, filed on Nov. 9, 2005.
(60) Provisional application No. 60/742,436, filed on Dec. 5, 2005.
(60) Provisional application No. 60/736,786, filed on Nov. 15, 2005.
(60) Provisional application No. 60/735,011, filed on Nov. 9, 2005.
(60) Provisional application No. 60/742,436, filed on Dec. 5, 2005.
(60) Provisional application No. 60/759,255, filed on Jan. 13, 2006.
(60) Provisional application No. 60/814,340, filed on Jun. 16, 2006.
(60) Provisional application No. 60/829,639, filed on Oct. 16, 2006.
(60) Provisional application No. 60/762,675, filed on Jan. 27, 2006.

(51) Int. Cl. A61M 31/00; A61M 37/00

(52) U.S. Cl. 604/500; 604/23

(57) ABSTRACT

The present invention provides an applicator for directing a flow of medically pure, therapeutic gas onto a selected portion of the body of a patient. The applicator receives the flow of medically pure, therapeutic gas, such as oxygen, from a source, via a conduit coupled to the source and to the applicator. The applicator can be an adhesive bandage having an inlet for receiving the flow of gas, and one or more outlets for directing the flow of gas. The applicator can also be a mask, patch or similar article configured to be applied to the skin of a patient and connected to a source of medically pure, therapeutic gas via a conduit. The applicator can also having other therapeutic materials impregnated therein. The applicator can also be configured to deliver other therapeutic substances introduced into the flow of gas.

### CROSS-REFERENCED APPLICATIONS

This application relates to, and claims the benefit of the filing date of, co-pending U.S. provisional patent application Ser. No. 60/735,011 entitled “OXYGEN PATCH,” filed Nov. 9, 2005, the entire contents of which are incorporated herein by reference for all purposes. This application also relates to the following co-pending U.S. patent applications, the entire contents of which are incorporated herein by reference for all purposes.
1. Ser. No. 10/718,131 entitled Method & Apparatus for Generating Oxygen, filed Nov. 20, 2003;
2. Ser. No. 10/856,591 entitled Apparatus and Delivery of Medically Pure Oxygen, filed May 28, 2004;
3. Ser. No. 11/045,805 entitled Method and Apparatus for Controlled Production of a Gas, filed Jan. 28, 2005;
4. Ser. No. 11/158,993 entitled Method and Apparatus for Controlled Production of a Gas, filed Jun. 22, 2005;
5. Ser. No. 11/159,016 entitled Method and Apparatus for Controlled Production of a Gas, filed Jun. 22, 2005;
6. Ser. No. 11/158,377 entitled Method and Apparatus for Controlled Production of a Gas, filed Jun. 22, 2005;
7. Ser. No. 11/158,362 entitled Method and Apparatus for Controlled Production of a Gas, filed Jun. 22, 2005;
8. Ser. No. 11/158,618 entitled Method and Apparatus for Controlled Production of a Gas, filed Jun. 22, 2005;
9. Ser. No. 11/158,989 entitled Method and Apparatus for Controlled Production of a Gas, filed Jun. 22, 2005;
10. Ser. No. 11/158,696 entitled Method and Apparatus for Controlled Production of a Gas, filed Jun. 22, 2005;
11. Ser. No. 11/158,648 entitled Method and Apparatus for Controlled Production of a Gas, filed Jun. 22, 2005;
12. Ser. No. 11/159,079 entitled Method and Apparatus for Controlled Production of a Gas, filed Jun. 22, 2005;
13. Ser. No. 11/158,763 entitled Method and Apparatus for Controlled Production of a Gas, filed Jun. 22, 2005;
14. Ser. No. 11/158,865 entitled Method and Apparatus for Controlled Production of a Gas, filed Jun. 22, 2005;
15. Ser. No. 11/158,958 entitled Method and Apparatus for Controlled Production of a Gas, filed Jun. 22, 2005;
16. Ser. No. 11/158,867 entitled Method and Apparatus for Controlled Production of a Gas, filed Jun. 22, 2005;
17. Ser. No. 60/699,094 entitled Method and Apparatus for Generating Oxygen, filed Jul. 14, 2005;
18. Ser. No. 60/742,436 entitled Flexible Reaction Chamber with Frangible Seals and activation methods, filed Dec. 5, 2005;
19. Ser. No. 60/736,786 entitled Method and Apparatus for Delivering Oxygenated Heated Vapor in Skin Care Applications, filed Nov. 15, 2005;
20. Ser. No. 60/735,011 entitled Oxygen Patch, filed Nov. 15, 2005;
21. Ser. No. 60/742,436 entitled Flexible Reaction Chamber with Frangible Seals and Activation Methods filed Dec. 5, 2005;
22. Ser. No. 60/759,255 entitled Method and Apparatus for Providing Improved Availability of Breathable Air in a Closed Circuit filed Jan. 13, 2006;
23. Ser. No. 60/814,340 entitled Method and Apparatus for Providing Improved Availability of Breathable Air in a Closed Circuit filed Jun. 16, 2006;
24. Ser. No. 60/829,639 entitled Method and Apparatus for Providing Improved Availability of Breathable Air in a Closed Circuit filed Oct. 16, 2006; and
25. Ser. No. 60/762,675 entitled Expandable Housing Generator filed Jan. 27, 2006.

### BACKGROUND OF THE INVENTION

1. Field of the Invention

The present invention relates to delivering a therapeutic substance and, more particularly, to a method and apparatus for delivering a therapeutic gas to a patient.

2. Description of the Related Art

The healing effects and cosmetic benefits of enriched oxygen applied to the human skin are widely accepted and are supported by recent studies. Many skin care products such as creams and potions claim to have oxygen contained therein, and while these products proclaim the benefits of the oxygen content, the oxygen content is actually miniscule thereby offering little of the beneficial effects of oxygen. Medically pure (USP) oxygen has not traditionally been accessible to the consumers, primarily because it is obtained by prescription, and it is usually obtained as a compressed gas in a pressurized tank. Oxygen obtained as a compressed gas in a pressurized tank is usually associated with explosion hazards and other dangers, high initial cost, irritation of air passages due in part to the very low moisture content in the compressed gas, and a significant intimidation factor. In addition to compressed gas oxygen tanks, there are solid state oxygen generating technologies commercially available. However, these solid state oxygen generating technologies require the use of potentially hazardous chemicals such as hydrogen peroxide and heavy metals, or present burn hazards from dangerously high temperatures as a result of the extreme exothermic reactions involved.

### SUMMARY OF THE INVENTION

The present invention provides an applicator for directing a flow of medically pure, therapeutic gas onto a selected portion of the body of a patient. The applicator receives the flow of medically pure, therapeutic gas, such as oxygen, from a source, via a conduit coupled to the source and to the applicator. The applicator can be an adhesive bandage having an inlet for receiving the flow of gas, and one or more outlets for directing the flow of gas. The applicator can also be a mask, patch or similar article configured to be applied to the skin of a patient and connected to a source of medically pure, therapeutic gas via a conduit. The applicator can also having other therapeutic materials impregnated therein. The applicator can also be configured to deliver other therapeutic substances introduced into the flow of gas.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 a block diagram of an apparatus for delivering a medically pure, therapeutic gas to a patient;

FIGS. 2A-2B is cross-sectional views of an applicator according to one aspect of the present invention.

FIGS. 3A-3B are plan views of an applicator illustrating the internal configuration thereof;

FIG. 4A is a cross-sectional view of an applicator according to another aspect of the present invention, and FIGS. 4B-4C are plan views of an applicator illustrating flow control techniques;

FIG. 5 is a simplified perspective view of an applicator illustrating another flow control technique;

FIG. 6A-6B are cross-sectional view of an applicator illustrating additional flow control techniques according to another aspect of the present invention;

FIGS. 7A-7C are plan views of an applicator illustrating alternative flow control techniques;

FIG. 8 is a simplified block diagram of an apparatus for delivering an additive mixed with a medically pure, therapeutic gas to a patient;

FIG. 9 is a simplified block diagram of an apparatus for delivering an additive-laden mist mixed with a medically pure, therapeutic gas to a patient;

FIGS. 10A-10B are cross-sectional views of an applicator illustrating alternative additive techniques according to another aspect of the present invention;

FIG. 11A-11B are cross-sectional views of an applicator illustrating the application of medicinal dressing materials according to another aspect of the present invention;

FIGS. 12A-12B are pictorial views of an applicator for delivering a therapeutic gas to a patient according to another aspect of the invention;

FIG. 13 is a block diagram of an apparatus for delivering a medically pure, therapeutic gas to a patient according to another aspect of the invention;

FIG. 14 is a block diagram of an apparatus for delivering a medically pure, therapeutic gas and electro-stimulation to a patient according to another aspect of the invention; and

FIG. 15 is a cross-sectional view of an applicator according to another aspect of the present invention.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail.

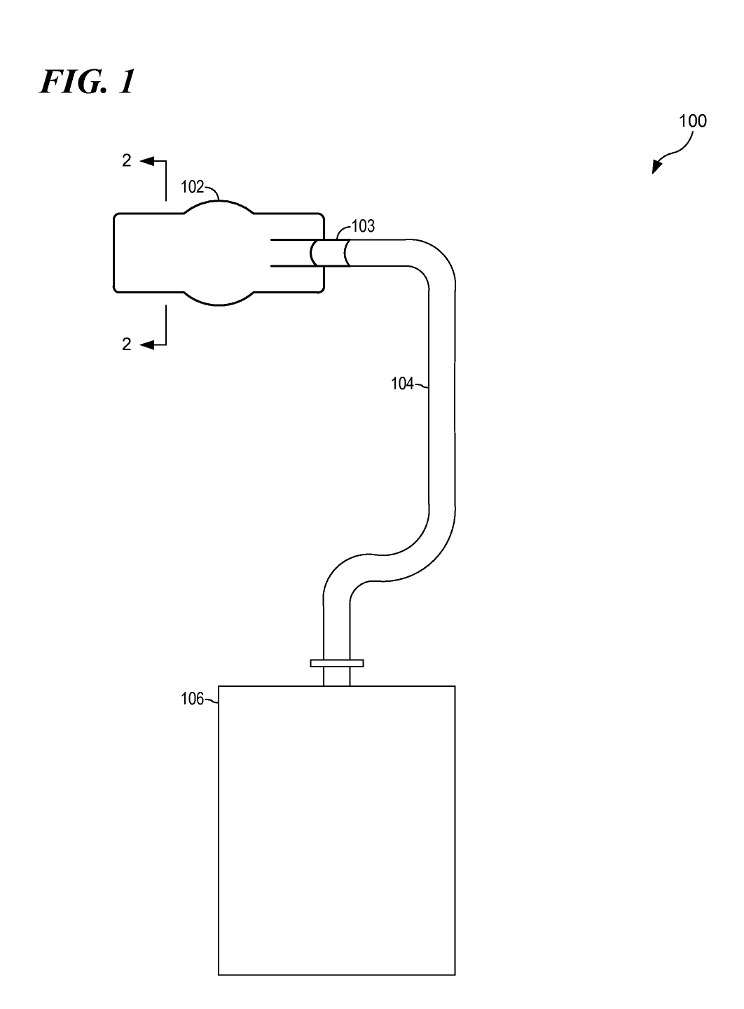

Turning now to FIG. 1, the reference numeral 100 generally indicates an apparatus for delivering a medically pure, therapeutic gas to a patient. The apparatus 100 comprises an applicator 102 having an inlet 103 which can be coupled to a conduit 104, which in turn can be coupled to a source 106 of medically pure, therapeutic gas. The applicator 102 can be applied directly to the skin of a patient for topical, external application of the therapeutic gas to a localized area, and removably secured in-place by a variety of techniques including, for example, the use of adhesives, straps, bandages, resilient materials and the like. The conduit 104 can be a flexible hose or tube coupled between the applicator 102 at inlet 103 and the source 106. The source 106 can be a pressurized tank of compressed gas, such as oxygen. The source 106 can also be a catalytic reaction chamber operating at a relatively low temperature and pressure, such as the apparatus and methods described in one or more of the following co-pending patent applications (collectively referred to as “Reaction Devices and Processes”), the entire contents of which are incorporated herein by reference for all purposes:
1. Ser. No. 10/718,131 entitled Method & Apparatus for Generating Oxygen, filed Nov. 20, 2003;
2. Ser. No. 10/856,591 entitled Apparatus and Delivery of Medically Pure Oxygen, filed May 28, 2004;
3. Ser. No. 11/045,805 entitled Method and Apparatus for Controlled Production of a Gas, filed Jan. 28, 2005;
4. Ser. No. 11/158,993 entitled Method and Apparatus for Controlled Production of a Gas, filed Jun. 22, 2005;
5. Ser. No. 11/159,016 entitled Method and Apparatus for Controlled Production of a Gas, filed Jun. 22, 2005;
6. Ser. No. 11/158,377 entitled Method and Apparatus for Controlled Production of a Gas, filed Jun. 22, 2005;
7. Ser. No. 11/158,362 entitled Method and Apparatus for Controlled Production of a Gas, filed Jun. 22, 2005;
8. Ser. No. 11/158,618 entitled Method and Apparatus for Controlled Production of a Gas, filed Jun. 22, 2005;
9. Ser. No. 11/158,989 entitled Method and Apparatus for Controlled Production of a Gas, filed Jun. 22, 2005;
10. Ser. No. 11/158,696 entitled Method and Apparatus for Controlled Production of a Gas, filed Jun. 22, 2005;
11. Ser. No. 11/158,648 entitled Method and Apparatus for Controlled Production of a Gas, filed Jun. 22, 2005;
12. Ser. No. 11/159,079 entitled Method and Apparatus for Controlled Production of a Gas, filed Jun. 22, 2005;
13. Ser. No. 11/158,763 entitled Method and Apparatus for Controlled Production of a Gas, filed Jun. 22, 2005;
14. Ser. No. 11/158,865 entitled Method and Apparatus for Controlled Production of a Gas, filed Jun. 22, 2005;
15. Ser. No. 11/158,958 entitled Method and Apparatus for Controlled Production of a Gas, filed Jun. 22, 2005;
16. Ser. No. 11/158,867 entitled Method and Apparatus for Controlled Production of a Gas, filed Jun. 22, 2005;
17. Ser. No. 60/699,094 entitled Method and Apparatus for Generating Oxygen, filed Jul. 14, 2005;
18. Ser. No. 60/742,436 entitled Flexible Reaction Chamber with Frangible Seals and activation methods, filed Dec. 5, 2005;
19. Ser. No. 60/736,786 entitled Method and Apparatus for Delivering Oxygenated Heated Vapor in Skin Care Applications, filed Nov. 15, 2005;
20. Ser. No. 60/735,011 entitled Oxygen Patch, filed Nov. 15, 2005;
21. Ser. No. 60/742,436 entitled Flexible Reaction Chamber with Frangible Seals and Activation Methods filed Dec. 5, 2005;
22. Ser. No. 60/759,255 entitled Method and Apparatus for Providing Improved Availability of Breathable Air in a Closed Circuit filed Jan. 13, 2006;
23. Ser. No. 60/814,340 entitled Method and Apparatus for Providing Improved Availability of Breathable Air in a Closed Circuit filed Jun. 16, 2006;
24. Ser. No. 60/829,639 entitled Method and Apparatus for Providing Improved Availability of Breathable Air in a Closed Circuit filed Oct. 16, 2006; and
25. Ser. No. 60/762,675 entitled Expandable Housing Generator filed Jan. 27, 2006.

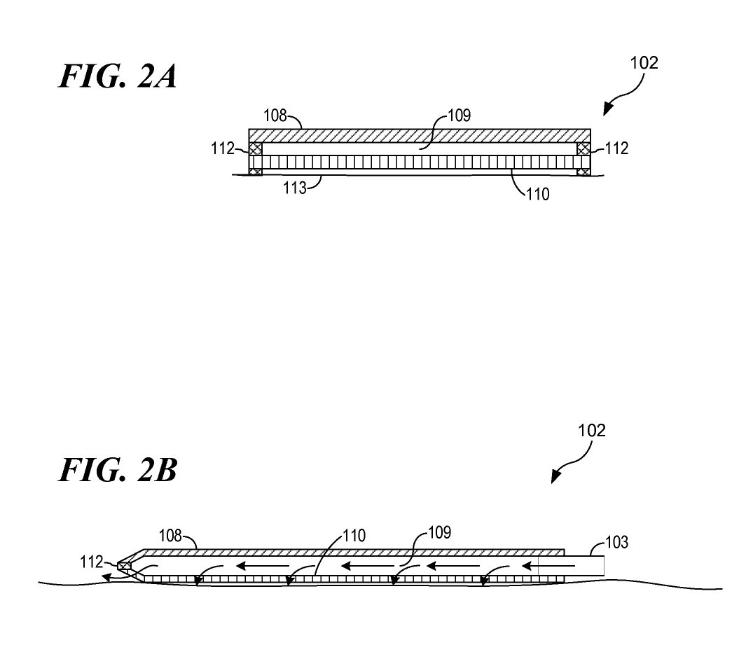

Turning now to FIGS. 2A-2B, and in certain embodiments of the invention, applicator 102 can be constructed as an adhesive bandage or patch that can be applied directly to the skin of a patient, covering a wound, damaged tissue or other area to be treated with a therapeutic gas, such as oxygen. The applicator 102 can be formed by adhering two layers of material 108, 110 with an adhesive 112, capturing an inlet tube 103 at one end thereof. A bottom layer 108, disposed closest to the skin when the applicator 102 applied, can consist of any porous material, membrane or other appropriate material that will allow the perfusion of a therapeutic gas, such as oxygen, from the applicator 102 to the skin. For example, bottom layer 110 can be a membrane with a 20 micron mesh size, or submicron mesh size, to prevent passage of viral elements. A top layer 108, disposed farthest from the skin when applicator 102 is applied, can consist of any barrier material that will prevent the oxygen flow from escaping, and thereby force the oxygen flow into the channels and through the bottom layer 110. For example, the top layer 108 can be made of polyethylene (PE), low density polyethylene (LDPE) or polyvinyl chloride (PVC) or various polymer or polymer and metal laminates. A space 109 between top layer 108 and bottom layer 110 serves as a channel for the flow of therapeutic gas, such as oxygen, from inlet 103 through bottom layer 110 to the skin. The underside of the bottom layer 110 can be coated with an adhesive similar to what can be found on a typical adhesive bandage in order to attach the patch 102 to the skin. This skin adhesive can be applied in patterns to form channels for dispersing the flow across the skin, and would be protected by a release film layer 113 until the time of application.

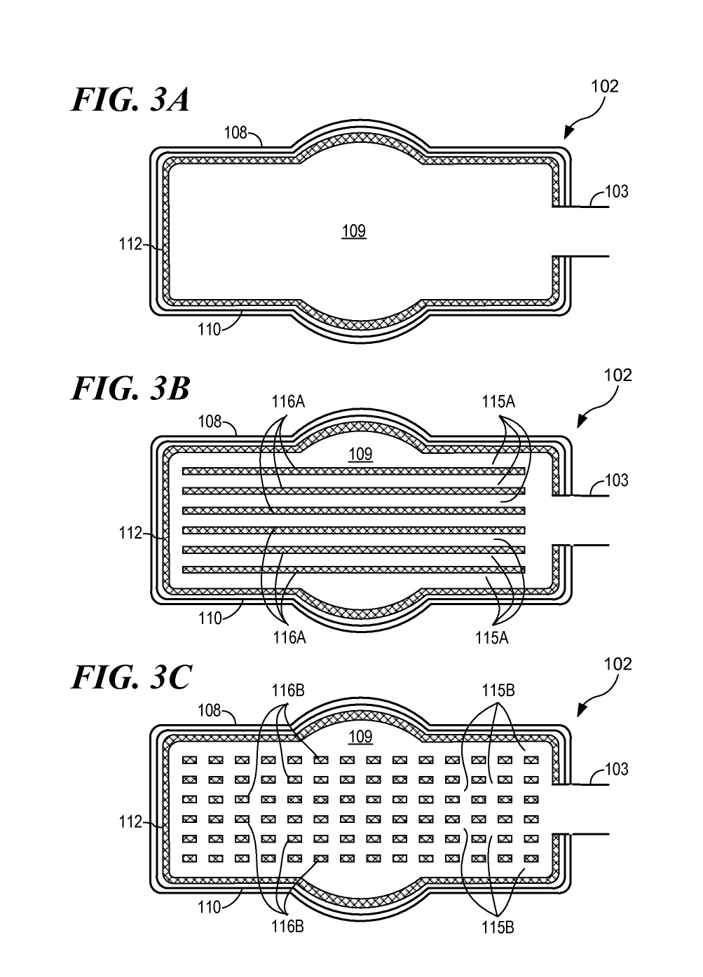

Turning now to FIG. 3A, in some embodiments applicator 102 can comprise the top layer 108 adhered by adhesive 112 to bottom layer 110 with inlet 103 secured therebetween, and wherein the space 109 is open and unobstructed to permit free flow of the therapeutic gas, such as oxygen from the inlet 103 to the skin. Applicator 102 can be designed to disperse the therapeutic gas over a desired area of the skin by numerous small channels. These channels disperse the oxygen flow across the desired skin treatment area, from where the therapeutic gas is absorbed into the skin and any other exposed body fluids and tissues.

Turning now to FIG. 3B, in some embodiments applicator 102 can comprise the top layer 108 adhered by adhesive 112 to bottom layer 110 with inlet 103 secured therebetween, and wherein the space 109 is divided into one or more channels 115 formed by further adhesive material 116A applied in substantially parallel rows on bottom layer 110 and adhering applicator 102 to the skin.

Turning now to FIG. 3C, in some embodiments applicator 102 can comprise the top layer 108 adhered by adhesive 112 to bottom layer 110 with inlet 103 secured therebetween, and wherein the space 109 is divided into a network of interconnecting channels 115B formed by further adhesive material 116B applied in a matrix of interstitial portions on bottom layer 110 and adhering applicator 102 to the skin.

A therapeutic gas, such as oxygen, can be generated using the Reaction Devices and Processes. In one embodiment, the oxygen source 106 is a flexible pouch that is squeezed or otherwise manipulated to commence the catalytic reaction, thereby commencing the oxygen flow via a tube or channel into the oxygen patch or low pressure reservoir. Other embodiments for oxygen source and activation of the process such as piercing containers or membranes, opening valves, and mechanical stirring, are numerous, yet all having the same purpose of initiation of the oxygen generating reaction through mixing of the reagents and activator.

After activation the oxygen flow is directed to the skin via conduit 104 to applicator 102 that is adhered to the skin, just like a Band-Aid® type adhesive strip bandage or otherwise held in place with other means such as but not limited to vacuum (suction), elastic or cloth bandages, or mechanical means such as straps or various tapes. For the adhesively attached patch, a protective material would be removed from the adhesive and the patch would be positioned over the desired area and lightly pressed in place. The applicator 102 can be made in standard sizes and can also be designed for customization by a user. Applicator 102 can prevent exhausting of the oxygen, or exhausting of the oxygen can be limited to maintain a positive pressure inside the applicator 102 during the treatment period in combination with some degree of flow across the treatment area. The oxygen flow can be laminar across the treatment area or turbulent with the option to impinge directly on the treatment area.

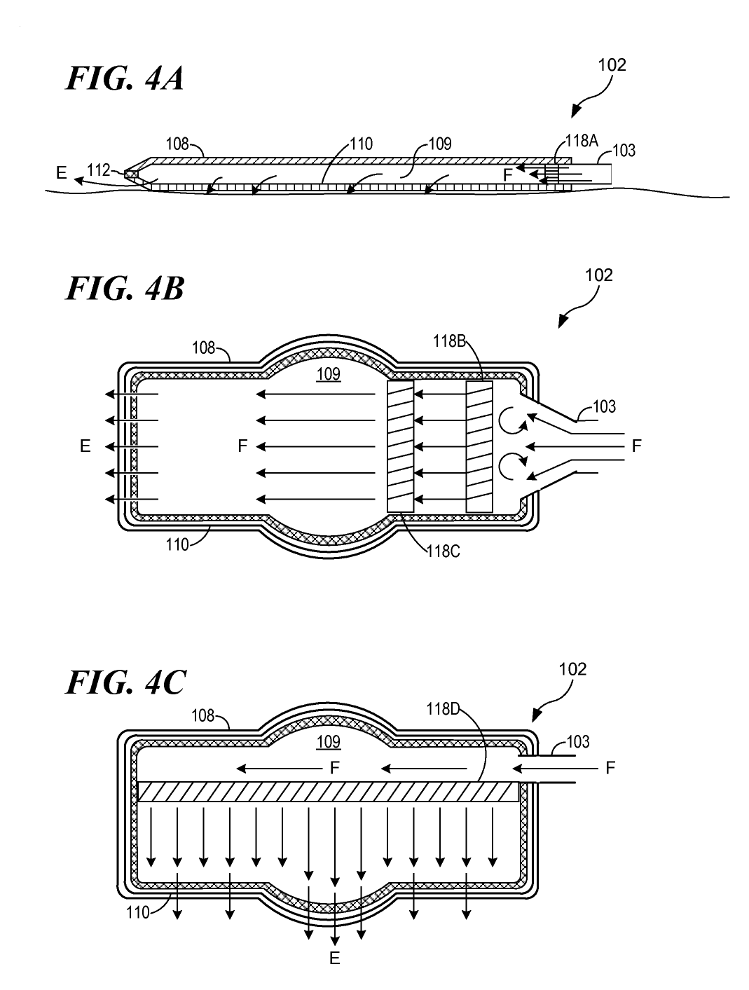

Turning now to FIGS. 4A-4C, oxygen flow can be directed by physical features so as to create a laminar flow of the oxygen across the treatment area. For example, and as shown in FIG. 4A, a structure 118A of honeycombed baffle materials placed at the inlet 103 of applicator 102 can transform turbulent flow into laminar flow. Alternatively baffles 118B and 118C can be placed within the space 109 between top layer 108 and bottom layer 110, as shown in FIG. 4B, to further direct the flow as indicated by arrows F. Orientation of the baffle(s) 118 can determine the direction of flow. As shown in FIG. 4C, a baffle 118D disposed longitudinally within the space 109 between top layer 108 and bottom layer 110 can direct laminar flow F transversely across the width of the applicator 102. The flow F can be exhausted through one or more edges of the applicator 102, as indicated by arrows E, to reduce pressure build-up within the space 109 and maintain flow F.

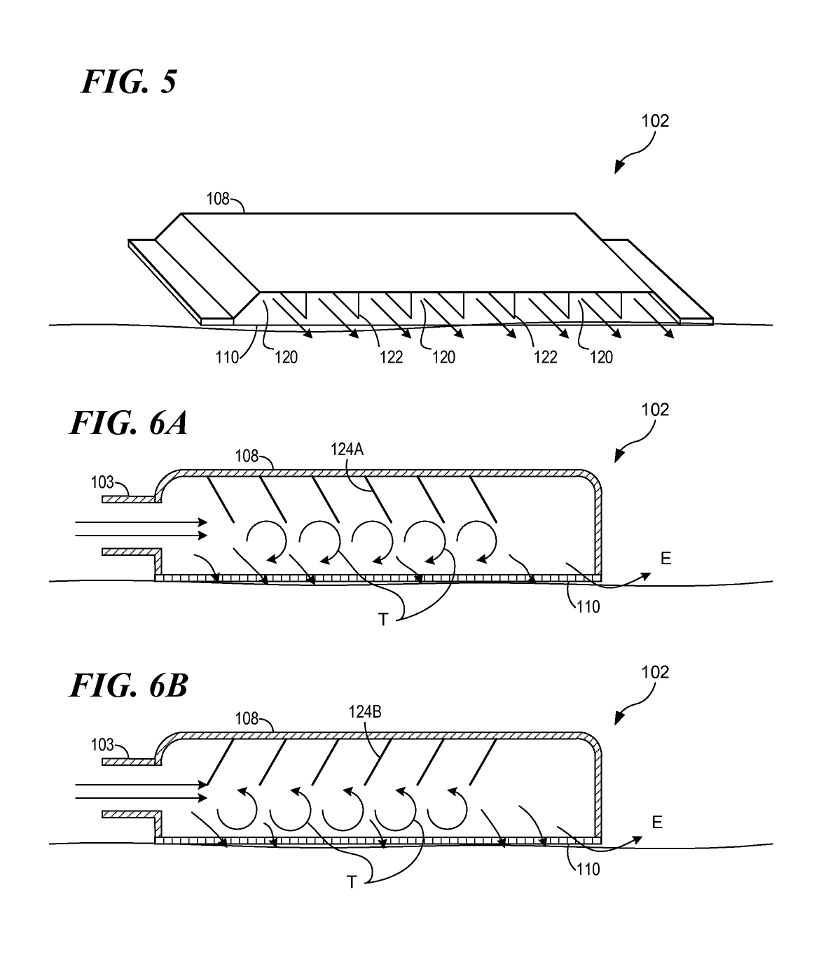

Turning now to FIG. 5, channels 120 can be added to the structure of applicator 102 to cause the flow to follow the laminar path. Channels 120 can be formed, for example, by baffle elements 122 disposed between top layer 108 and bottom layer 110.

Turning now to FIGS. 6A-6B, turbulent flow T can be accomplished by disruptive vanes or baffles 124 disposed between top layer 108 and bottom layer 110, partially blocking the flow and creating disruptions in the flow path that cause the gas to swirl, as depicted by curved arrows T.

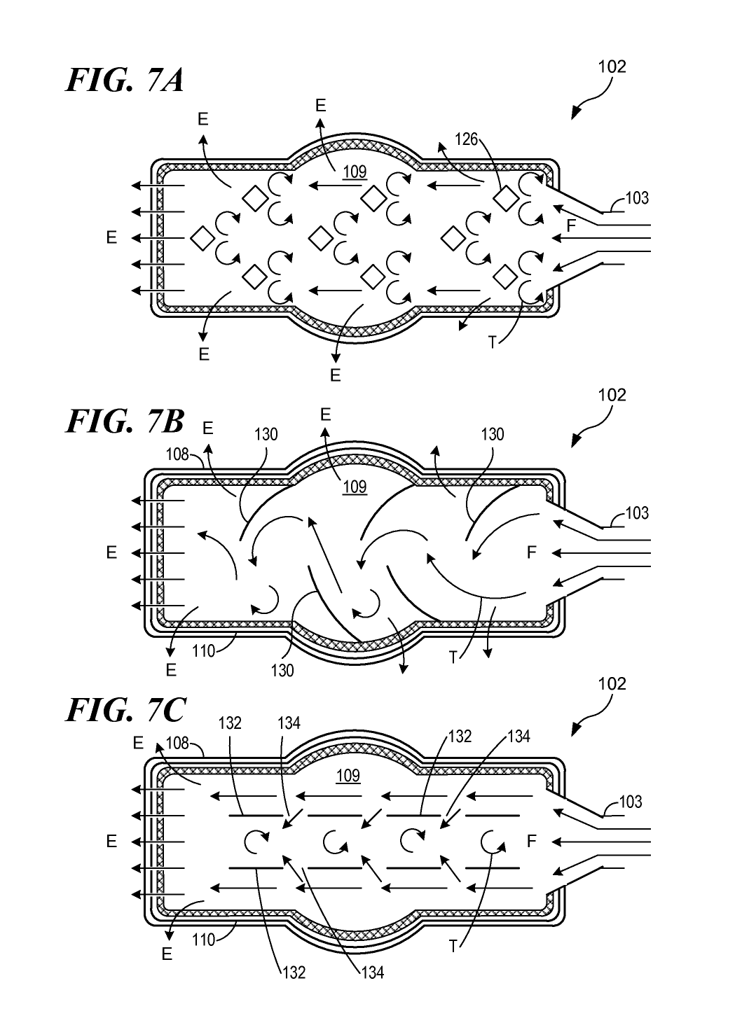

Turning now to FIGS. 7A-7C, disruptive baffles disposed between top layer 108 and bottom layer 110, partially blocking the flow and creating disruptions in the flow path can take additional forms. Geometrically-shaped obstructions 126 can be disposed within the space 109 between top layer 108 and bottom layer 110 to create disruptions in the flow, as shown for example in FIG. 7A. Vanes 130 can be disposed within the space 109 between top layer 108 and bottom layer 110 to create disruptions in the flow, as shown for example in FIG. 7B. Planar, spaced apart baffles 132 can be disposed within the space 109 between top layer 108 and bottom layer 110, having ports, gaps or apertures 134 between the baffles 132 to create disruptions in the flow, as shown for example in FIG. 7C. The flow F can be exhausted through one or more edges of the applicator 102, as indicated by arrows E, to reduce pressure build-up within the space 109 and maintain flow F.

If the source of the oxygen applied to the healing process is a pressurized tank of compressed gas, then the oxygen provided is generally a dry oxygen. The Reaction Devices and Processes provide alternative sources that enhance the application by providing moist oxygen. Percentages of water vapor in the oxygen can be controlled with various filter and vapor barrier materials as required by particular applications. Filter materials include but are not limited to, PTFE, PTFE coated materials such as polyester, nylon, polypropylene, and so on, PVDF, and many other commonly used polymers, carbon, and even metals. The features that make the filter materials useful as vapor barrier materials are hydrophobic properties, pore size, and or thermal capacity. This process generates a moist flow of oxygen. Moisture content can be controlled to an extent by condensing moisture out of the flow using hydrophobic filter materials with a small pore size in the range of 3 to 65 microns, or sub-micron sized pores can also be used. Filter elements or vapor barrier materials can create moisture traps and can be added to reduce the moisture content provided at the outflow.

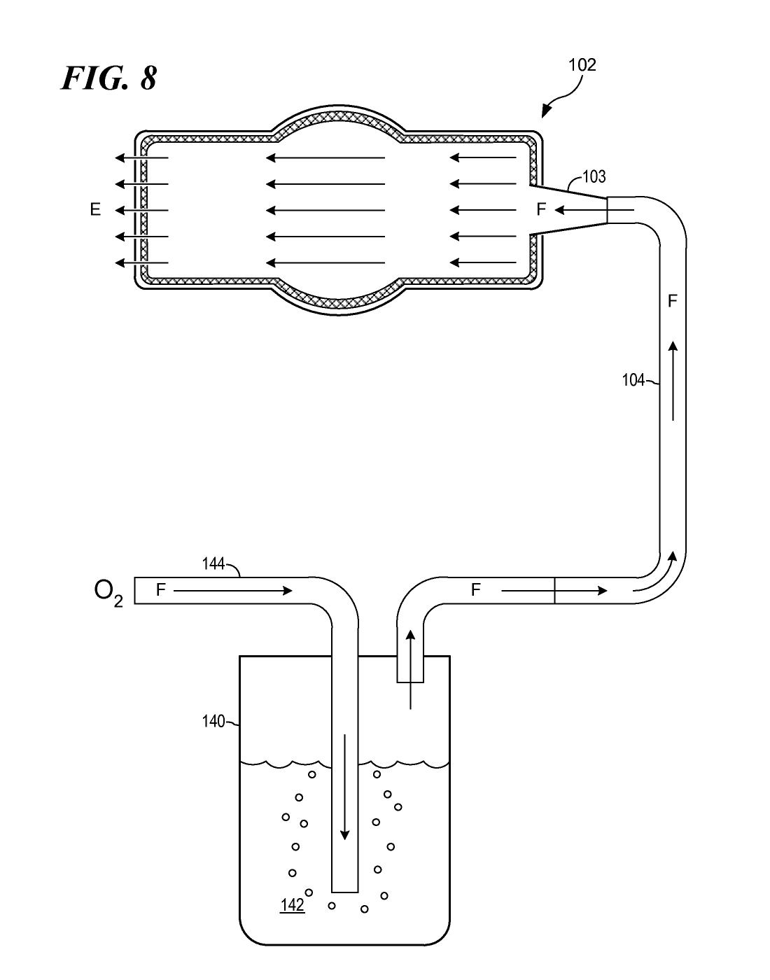

The low pressure of the Reaction Devices and Processes allow for the introduction of additional beneficial nutrients or medications as additives as desired. These additives can include but are not limited to nutraceuticals, pharmaceuticals, anti-biotic and anti-microbial agents, essential oils, vitamins and/or scents. These agents can be introduced as part of the applicator 102 or introduced into the oxygen flow or both. Additives can be introduced in many ways. Turning now to FIG. 8, additives can be introduced into the flow F using a bath 140 containing the additive or additive solution 142, which can be a liquid or a gel. Oxygen from the generator 106 is delivered via a conduit 144 to the additive bath 140 and, as the gas bubbles through additive solution 142, it picks up some portion of the additive and carries it forward in the oxygen stream, via conduit 104 to applicator 102 at inlet 103.

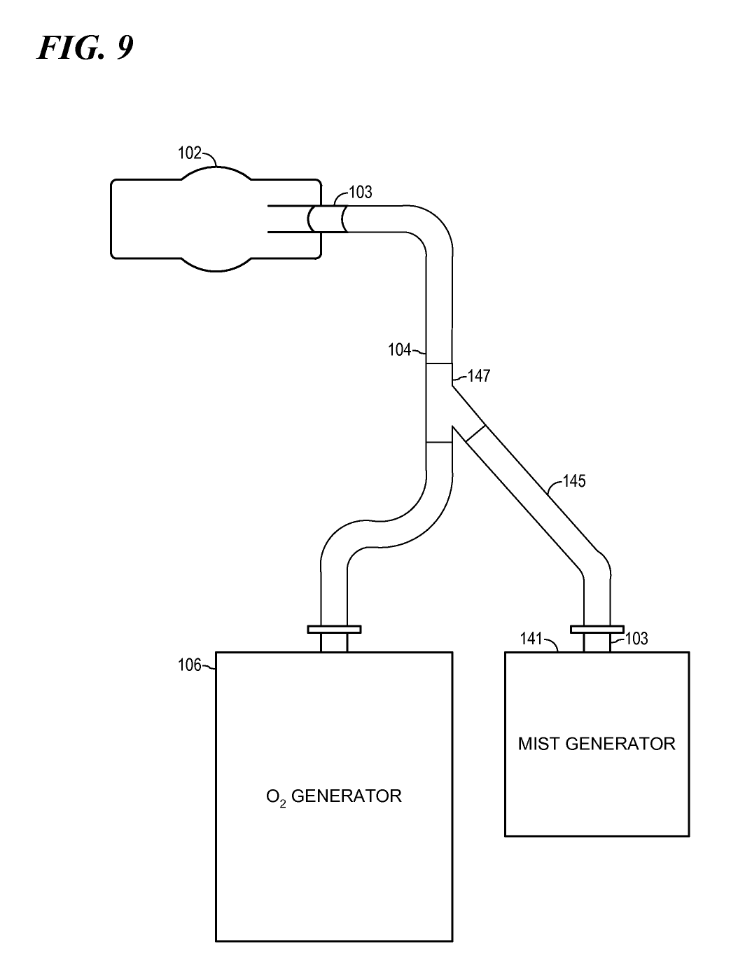

Turning now to FIG. 9, another method would include introducing an additive-laden mist into the oxygen stream. A mist generator 141 having an output 143 can be coupled to a conduit 104 via a conduit 145 and a coupling or manifold 147 to introduce an additive-laden mist into the oxygen stream. An additive-laden mist can be generated, for example, by the apparatus described in U.S. Provisional Patent Application No. 60/736,786 filed Nov. 15, 2005, and entitled “METHOD AND APPARATUS FOR DELIVERING OXYGENATED HEATED VAPOR IN SKIN CARE APPLICATIONS”, which is hereby incorporated by reference for all purposes.

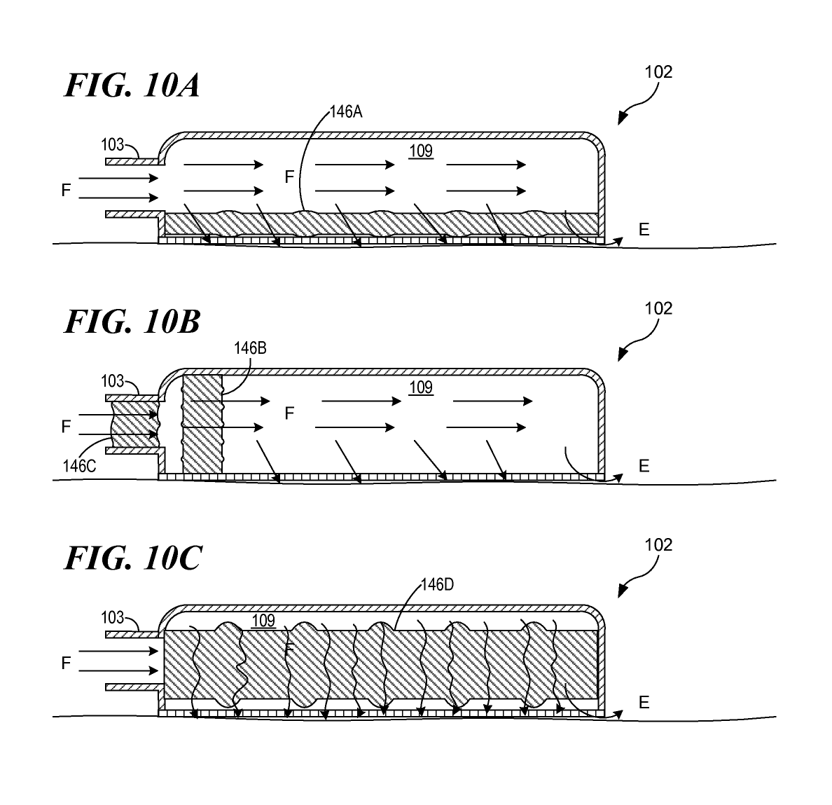

Turning now to FIGS. 10A-10C, additive carriers such as evaporative solids and/or impregnated membranes or support materials 146A, 146B, 146C, 146D can be placed in the oxygen flow to transfer additives in combination with the oxygen for introduction onto the treatment area. Permeable, additive-laden materials can be oriented in almost any direction and combined with a cavity on front and/or back of the membrane and or can completely or substantially fill the space 109 within applicator 102, thereby placing the additive in the flow F so that the additive will be carried to the treatment area by the gas flow to allow a combination of the beneficial effects of oxygen and the desired additive. Additive carriers can be placed within applicator 102 in the space 109 between top layer 108 and bottom layer 110. As shown in FIG. 10A, additive carrier 146A can be disposed within space 109 and adjacent to bottom layer 110. As shown in FIG. 10B, additive carrier 146B can be disposed within space 109 and proximal to inlet 103. Additionally or alternatively, additive carrier 146C can be disposed within inlet 103, as shown in FIG. 10B. An additive carrier such as 146C can also be disposed anywhere else along the conduit 104 between applicator 102 and generator 106 shown in FIG. 1. As shown in FIG. 10C, an additive carrier 146D can be disposed within applicator 102 and substantially filling the space 109.

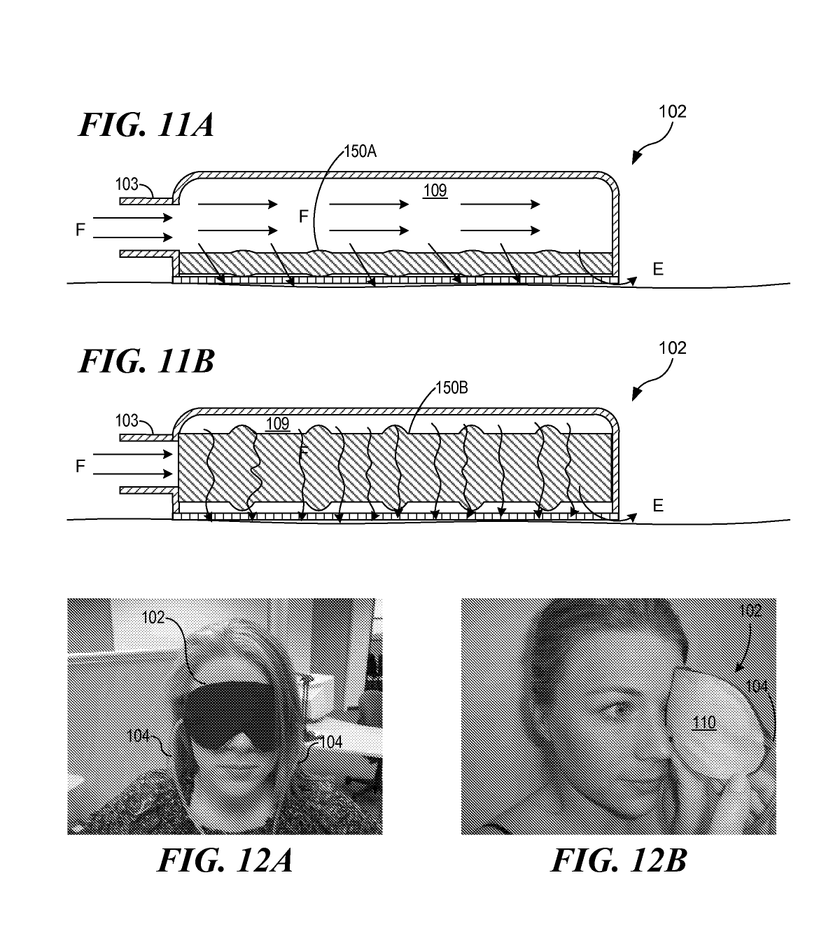

Turning now to FIGS. 11A-11B, where the condition of the treatment area will allow contact, permeable materials treated or impregnated with a desired therapeutic additive can be applied with the applicator 102. Multiple beneficial effects can be obtained by combining oxygen treatment in combination with the benefits of additives. The applicator 102 may contain materials typically used in wound dressing and in such cases the applicator 102 can direct the flow of oxygen through the dressing materials to the wound utilizing flow control techniques as described above. Dressing materials can be infused with additional beneficial materials such as would cleanse the wound or otherwise enhance the healing process. For example vitamins A, E, and D can be impregnated into the dressing materials. Pain relief medications could also be delivered via the applicator 102. Direct application of the medication or additive to the treatment area followed by application of oxygen treatment via applicator 102 is also envisioned. Thereby allowing the benefits of combining additives and high purity oxygen without having to have an inventory of patches with specific medications. The applicator 102 can be used with a medicated or impregnated insert 150. As shown in FIG. 11A, a medicated insert 150A can be a thin membrane that is permeable to the therapeutic gas such as oxygen, having a cavity impregnated with a medication, and in contact with a treatment area when applicator 102 is applied thereto. As shown in FIG. 11B, a medicated insert 150B can be a thicker membrane that is permeable to the therapeutic gas such as oxygen, having a cavity impregnated with a medication and substantially filling the space 109 within applicator 102, and in contact with a treatment area when applicator 102 is applied thereto.

Turning now to FIGS. 12A-12B, the applicator 102 can also take the form of mask or like article for covering a selected treatment area of a patient's skin. As shown in FIG. 12A, the mask applicator covers a portion of a patient's face when applied, and delivers a therapeutic gas, such as oxygen, to the selected area via one or more flexible conduits 104 in one or more of the ways described above. As shown in FIG. 12B, a bottom layer 110 contacts the selected area of the patient's skin, and delivers the therapeutic gas through a membrane with an appropriate mesh size for minimizing the passage of viral elements.

This invention provides a portable oxygen system that generates oxygen catalytically in a safe manner without the hazards of compressed gas, with a low operating temperature, is inherently easy to operate, and is inexpensive to acquire. The system can easily be operated in a home use or consumer environment. Byproducts of the reaction contain no heavy metals and are benign. In various embodiments, the oxygen source includes the Reaction Devices and Processes. This allows for topical oxygen therapy to be consumerized and suitable for personal or home use. However any oxygen source, including a compressed tank, a liquid oxygen source or a concentrator can be used, given the users are cognizant of, trained in, and follow the proper safety precautions when dealing with the hazards associated with these other sources. Utilizing the Reaction Devices and Processes, the current invention can generate a given volume of oxygen per source volume of reactant. Small volumes of oxygen can be used for single use temporary or disposable patches and larger volumes for delivery to multiple users or for low pressure storage. Flow rates and oxygen volumes are adjustable and can be tailored for specific applications. The oxygen flow rate and the volume of oxygen produced can be tailored by manipulating a number of variables including but not limited to: particle size, powder formulation, volume of powder, volume of catalyst, volume of accelerator, catalyst formulation, and temperature. Various coatings can be applied to the powders to slow the reaction utilizing techniques described in the above-referenced and incorporated patent applications.

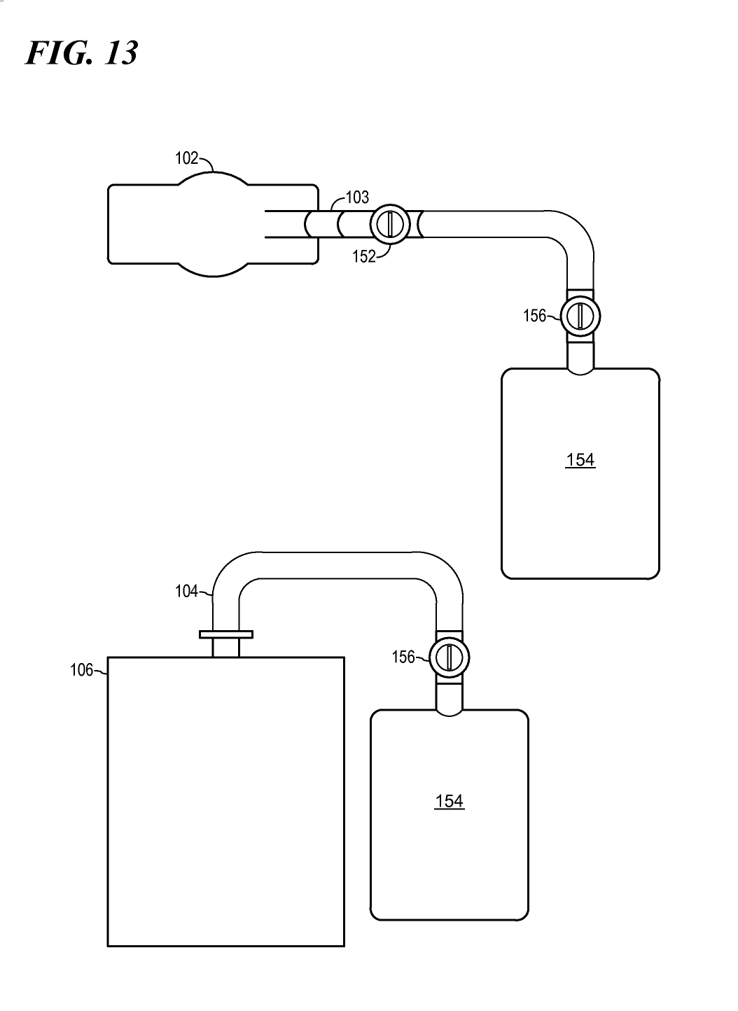

Turning now to FIG. 13, in another embodiment the applicator 102 can be fitted with a re-sealable valve or fitting 152 for multiple uses by one or multiple users. A larger generator package 106 can generate oxygen which can flow into a low pressure reservoir 154 for storage. The user can then attach the inlet 103 of applicator 102 to the reservoir 154 to replenish the oxygen in the applicator 102 according to a treatment schedule. The reservoir 154 can be as simple as an expandable oxygen barrier bag that can be pressed or squeezed to provide the force needed to cause oxygen flow into the patch. A valve 156 can also be attached to the reservoir. It would be possible to cleanse and sterilize this patch for multiple uses by an individual or in home use much the same as other oxygen delivery systems. The re-sealable valve or fitting can as simple as a Schrader valve such as used on bicycles but numerous other re-sealable valves are known and could be used. They use spring force or other means of a closing force to close the valve members against O-rings and seats to retain the gas. Other designs use o-rings and threads to activate the openings. Guillotine valves are another example.

Turning now to FIG. 15, means can be incorporated for active circulation of the oxygen held in a sealed applicator 102 such as manipulation of the outer covering as a diaphragm, or by using a powered fan or piezoelectric mechanism 170 disposed within the applicator 102, for example; within the inlet 103. One embodiment can circulate the flow passively through temperature gradients, body movement, or other passive means. Where power is available to activate electronic devices, fans and piezoelectric mechanisms can be implemented to actively move the oxygen atmosphere to increase the flow rate across the treatment area. This can provide a cooling effect and increase the interaction of the oxygen with the treatment area. Micro-miniaturized fans can be used. Piezoelectric materials deform when activated with an electrical charge. This can create the effect of a diaphragm or create movement similar to a flag waving. Feature sizes of these mechanisms are as small as the nano scale. Nano forests can be manipulated to create waves across the surface to drive the air currents. Mechanical movements of these materials can create movement of the oxygen inside the patch.

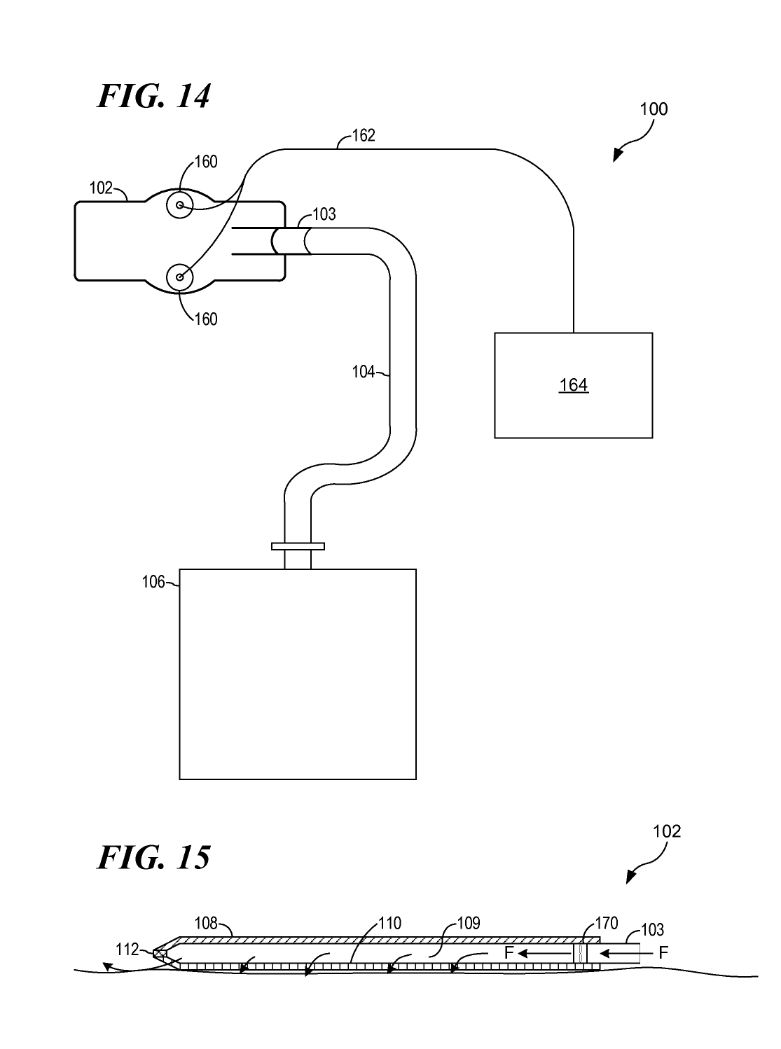

Electro stimulation techniques can be combined with the therapeutic gas applicator 102 to combine healing techniques. The treatments can be conducted alternately or in combination provided adequate precautions are taken to prevent any sparks or combustion hazards. Electro-stimulation is being employed for chronic wound healing. The beneficial effects of oxygen treatments using the system of the present invention can be combined with the electrical stimulation procedures. For example, chronic wound healing is a significant problem for diabetics. Problem with circulation in the extremities results in low oxygen levels in the tissues and is a contributing factor in the slow rate of healing. The system of the present invention can provide the needed oxygen and can further speed the healing process when combined with the proper electro-stimulation. Turning now to FIG. 14, and applicator 102 having an inlet 103 for coupling to a conduit 104 conveying a therapeutic gas, such as oxygen, from a generator 106, includes electrodes 160 for providing electro-stimulation in combination with therapeutic gas treatment. The electrodes 160 can be connected by electrical conductors 162 to a source 164 of electro-stimulation signals. Patients can receive stimulation with any kind of acceptable form, in cases of treatment for diabetes or open ulcers for example; an asymmetric biphasic (A) or symmetric biphasic (B) square-wave pulse. Amplitudes can be set to activate intact peripheral nerves in the skin.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

We claim:

1. An apparatus for delivering a therapeutic gas to a patent, comprising:
a source capable of generating a flow of medically pure, therapeutic gas;
a conduit coupled to the source, for conveying the flow of medically pure, therapeutic gas to the patient; and
an applicator, coupled to the conduit for receiving the flow of medically pure, therapeutic gas from the source, the applicator configured to direct the flow of medically pure, therapeutic gas into contact with a selected portion of the patient.

2. The apparatus of claim 1, wherein the applicator comprises an adhesive bandage having an inlet for receiving the flow of medically pure, therapeutic gas and one or more outlets directing the flow of medically pure, therapeutic gas into contact with a selected portion of the patient.

3. The apparatus of claim 1, wherein the applicator comprises a mask having an inlet for receiving the flow of medically pure, therapeutic gas and one or more outlets directing the flow of medically pure, therapeutic gas into contact with a selected portion of the patient.

4. A method for delivering a therapeutic gas to a patent, comprising the steps of:
generating a flow of medically pure, therapeutic gas;
conveying the flow of medically pure, therapeutic gas to the patient; and
directing the flow of medically pure, therapeutic gas into contact with a selected portion of the patient.

5. The method of claim 4, further comprising the step(s) of receiving the flow of medically pure, therapeutic gas from the conduit to an applicator and directing the flow of medically pure, therapeutic gas through one or more outlets of the applicator and into contact with a selected portion of the patient.
